(19) United States
(12) Patent Application Publication
(10) Pub. No.: US 2010/0305406 A1
(43) Pub. Date: Dec. 2, 2010

(54) SYSTEM, DEVICE AND METHOD FOR GYNECOLOGICAL USE

(76) Inventors: Ori BRAUN, Palo Alto, CA (US); Elisha Rabinovitz, Haifa (IL)

Correspondence Address: Pearl Cohen Zedek Latzer, LLP, 1500 Broadway, 12th Floor, New York, NY, 10036, US

(21) Appl. No.: 12/787,143

(22) Filed: May 25, 2010

Related U.S. Application Data
(60) Provisional application No. 61/180,994, filed on May 26, 2009.

(51) Int. Cl. A61B 1/32

(52) U.S. Cl. 600/202

(57) ABSTRACT

A device for gynecological examination has an imaging unit, which may include an illumination source, an imaging unit and an optical system, positioned within a speculum type device, typically between the blades thereof. The imaging unit is positioned at the end of an elongated rod that is positioned within a guide tube, both of which pass from between the blades down through the speculum handle, where they can be manipulated by a user to push the unit upward and forward for better viewing. A transmitter for transmitting sensed data to an external or remote receiver may be part of or may be separate from the device. The device may have non-imaging sensors, such as a pH sensor, a sensor to sense electrical impedance of tissues, a temperature sensor, etc., either by itself or in addition to an imager. A system may further include a remote receiver, a processor for processing data and a monitor for presenting images to the user in real time.

### CROSS-REFERENCE TO RELATED APPLICATION

This application claims the benefit of U.S. Provisional Patent Application No. 61/180,994, filed May 26, 2009.

### FIELD OF THE INVENTION

The present invention relates to system, device and method for gynecological examination. More specifically, the present invention relates to a system, device and method for viewing and examining the cervix and related anatomy.

### BACKGROUND OF THE INVENTION

Colposcopy is the standard tool in the United States for diagnosing cervical abnormalities after an abnormal pap smear. The main goal of colposcopy is to prevent cervical cancer by early detection and treatment of precancerous lesions.

Colposcopy is a medical diagnostic procedure to examine the cervix and tissues of related anatomy such as the vagina and vulva. Premalignant lesions and malignant lesions in these areas have discernible characteristics that can be detected thorough the examination. Colposcopy involves viewing of the cervix, vagina, and vulva through a high-powered microscope called a colposcope. The colposcope consists of large, electric microscope, often in the form of binoculars, attached to a stand and an illuminating unit. Direct examination through the colposcope allows the detection of abnormalities on the cervix that can not be seen with the naked eye.

During the colposcopy procedure, a vaginal speculum (typically consisting of two hinged parts or blades that together form a hollow cylinder with a rounded end) is used to hold the vaginal cavity open in order to allow viewing of the cervix. Acetic acid solution (and sometimes also Iodine) is applied to the cervix and vagina to highlight areas (tissue which is thickened, such as cancer cells) by turning them white (instead of a normal pink color). Abnormal areas can also be identified by looking for a characteristic pattern made by abnormal blood vessels, which may indicate new growth, such as cancer. Use of the colposcope provides an enlarged, illuminated view of the areas and enables taking biopsies for further pathological examination. Different magnifications may be used and various light filters are available to highlight different aspects of the surface of the cervix.

It is well known that during routine gynecological examination it is impossible to diagnose diseases or other problems simply by looking at the cervix with the naked eye. A magnified view is necessary in order to find any abnormalities or in order to show that cervical changes are not a cause for concern. A colposcope is also necessarily limited by the fact that it performs its examinations from outside the cervix. Furthermore, in order to insert the large microscope through the speculum, the speculum's hinged blades are required to be distanced from one another for quite a large distance, which might cause some inconvenience to the patient. In addition, during colposcopy, the physician holds the speculum in one hand while holding the microscope in his other hand, which makes it difficult to take a biopsy, for example, or to perform a pap smear during the colposcopy procedure. The colposcopy procedure, therefore, requires many resources and cumbersome equipment, and can be expensive to perform, making it a less-than-ideal screening tool.

Many attempts have been made heretofore to develop a simple and portable colposcopic apparatus that would allow for the ready use of the colposcope as a screening tool both outside and inside the cervix, thereby increasing the opportunity for diagnosis and treatment.

Therefore, it is desirable to provide a lightweight, portable and simple to use magnification apparatus useful as a colposcope.

It is desirable to provide an alternative colposcopic viewing instrument that is less cumbersome and safer to use than existing devices.

### SUMMARY OF THE INVENTION

It is an object of the present invention to provide a system, device and method for colposcopic viewing that is less cumbersome and safer to use than existing devices and allows viewing of tissue from both outside and inside the cervix.

In certain embodiments of the present invention, a system for viewing the cervix and related areas is provided. According to one embodiment, the system includes a speculum type device having an imaging unit attached thereto, said imaging unit capable of transmitting images to a remote receiver; a receiver typically positioned externally to the patient; and a processor for processing data received by the receiver. According to some embodiments, the system includes a monitor or other display for presenting images to the user. According to embodiments of the invention, the imaging unit transmits images of the areas under examination to a receiver external to the patient. The images may be transmitted wirelessly or through a cable to the remote receiver. The received images may be processed and presented, typically in real time, to the user.

According to some embodiments of the invention, a device for gynecological examination comprises a vaginal speculum having an upper blade with a distal end and a proximal end and having a lower blade with a distal end, a proximal end and a handle. The upper and lower blades have means on their respective proximal ends for moveably interconnecting the proximal ends for relative movement between the first and second blades. A sensing unit is movably positioned between the upper and lower blades, and a mechanism for enabling movement of the sensing unit relative to the upper and lower blades passes through the handle and has a distal end attached to the sensing unit and operates via remote actuation of the proximal end thereof.

Embodiments of the invention provide an imaging unit within the speculum type device to be used in gynecological examinations. Since the imaging unit is an integral part of the speculum, the imaging unit enables the physician to hold the speculum and the imaging unit with one hand, while freeing the other hand for performing various additional procedures, e.g., taking a biopsy from the cervix or performing pap smear. According to some embodiments, the imaging unit may include an illumination source, such as LEDs or other suitable illumination source, an imager and an optical system for focusing an image onto the imager. A transmitter for transmitting image data to an external or remote receiver may be part of the unit or may be separate from it.

According to some embodiments, the imaging unit may instead be a sensor unit which includes non-imaging sensors, such as a pH sensor, a sensor to sense electrical impedance of tissues, a temperature sensor, etc., either by itself or in addition to an imager. The transmitter may be used to transmit any sensed data, whether image data or other data.

In other embodiments, a system for gynecological examination has, in addition to the device described above, a receiver for receiving signals representing sensed data transmitted from said device and a means for presenting the sensed data of a body lumen to a user.

A method according to embodiments of the invention may include the steps of sensing parameters of the cervix and/or related areas, transmitting the sensed data to a receiver, processing the received data and presenting the processed data to a user. According to one embodiment, sensing includes imaging.

### BRIEF DESCRIPTION OF THE DRAWINGS

The above and other objects and advantages of the invention will be understood and appreciated more fully upon consideration of the following detailed description, taken in conjunction with the accompanying drawings, in which the reference characters refer to like parts throughout and in which:

FIG. 1 shows an overall schematic illustration of a system operative according to one embodiment of the present invention;

FIG. 2 shows a schematic illustration of a speculum and sensing unit according to one embodiment of the present invention;

FIGS. 3A-B depict devices operative according to embodiments of the present invention; and

FIG. 4 depicts an imaging unit according to an embodiment of the present invention.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

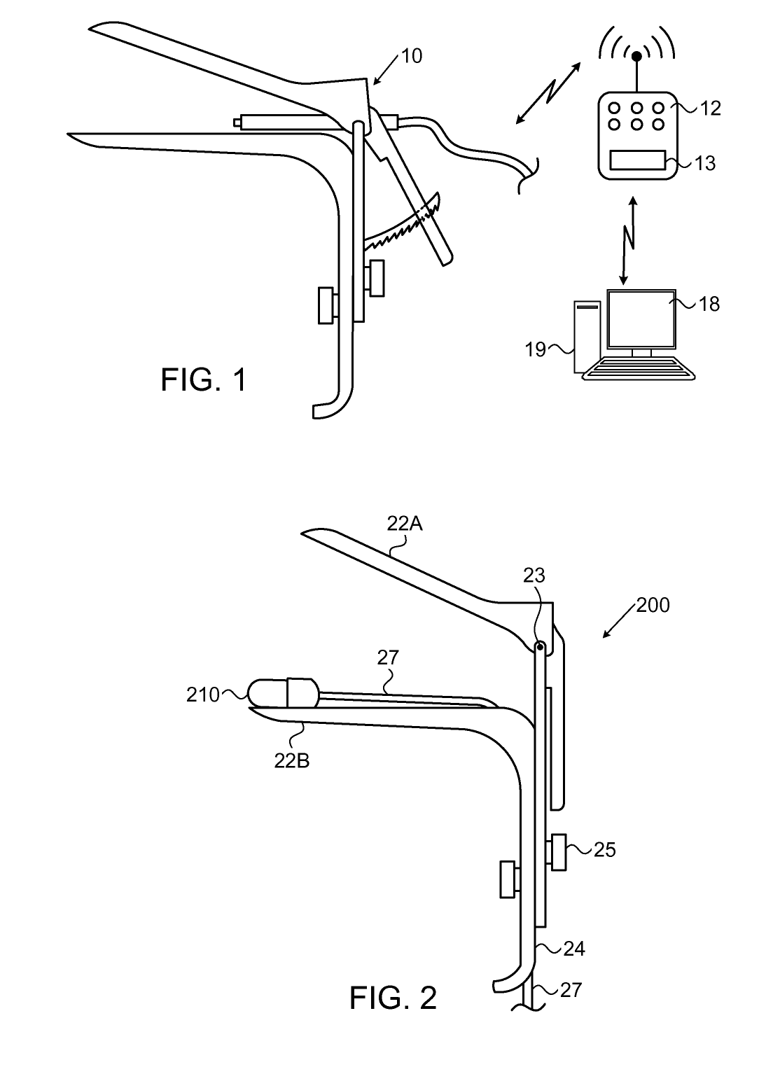

Reference is made to FIG. 1, which shows an overall schematic illustration of an exemplary system operative according to one embodiment of the present invention. The system includes a device 10, typically designed to comprise a speculum-like device, which is to be inserted into the vagina to dilate the vagina for examination of the vagina and cervix. The device 10 may be used to examine other body parts having an orifice whose interior requires examination, and the device 10 may have a form and shape that are dependent on the body cavity that it is designed to examine.

The device 10 has attached to it, or incorporated within it, a sensing device to sense (such as by imaging) the body orifice interior and a transmitter to transmit the sensed data to a receiver/recorder 12 and a data processing unit including a processor 13, which is typically located outside the patient's body in one or more locations. Transmission may be wireless or through a wired connection. Typically, the receiver 12 may be small and portable, and may be placed on or in the vicinity of the patient's body during recording of the data.

According to some embodiments, the receiver 12 may be part of or may be connected to a display or monitor 18. In some embodiments, data may be transmitted from receiver 12 to monitor 18 wirelessly or through a wired connection. According to some embodiments, monitor 18 can be part of a personal computer or workstation, which includes standard components such as a data processor or a data processor storage unit 19, a memory, a disk drive, and input-output devices, although alternate configurations are possible. The data processor may include any standard data processor, such as a microprocessor, multiprocessor, accelerator board, or any other serial or parallel high performance data processor. Data processor may typically, as part of its functionality, act as a controller that controls the display of the sensed data (such as images) received by receiver 12.

Monitor 18 may be a conventional video display, but may, in addition, be any other device capable of providing image or other data. The monitor 18 may present image data in the form of still and moving pictures, and in addition may present other information. In an exemplary embodiment, the various categories of information are displayed in windows. The monitor 18 may include action or control buttons and graphical aids to facilitate viewing of the data. For example, zooming in on images, marking images, comparing images to a pre-stored database and other such actions may be enabled through monitor 18. Multiple monitors may be used to display image and other data.

According to one embodiment, a user sets up the receiver 12 on or close to the patient's body and manipulates the device 10 within the patient's body while viewing monitor 18. For example, device 10 may include an imaging unit and a transmitter which transmits image data of the cervix. The image data is received, processed and then displayed, typically in real-time on monitor 18 for a physician to view while manipulating the device 10 in the patient's body.

According to some embodiments, the imaging unit operation or other aspects of the device 10 may be controlled by the user 30 through monitor 18 or through receiver 12 or automatically based on processing procedures within the receiver or monitor.

Reference is now made to FIG. 2, which is a schematic illustration of a speculum and sensing unit according to one embodiment of the present invention. The speculum, here referred to as device 200, has two blades or arms, upper arm 22a and lower arm 22b, which may be shaped as hollowed out half cylinders, connected by hinge 23, which enables the user to create a required angle between the two arms 22a and 22b to hold apart tissues of the body orifice being examined. The device 200 may include a handle 24, which is typically integral with lower arm 22b for the user to grasp while using the device 200. The handle 24 may include a mechanism 25 for manipulating arms 22a and 22b or other parts of the device 200, so as to raise or lower upper arm 22a with respect to lower arm 22b, to thereby widen the distance between arms 22a and 22b, and for securing them in that adjusted position. The vertical adjustment mechanism 25 may include buttons, knobs, bolts, grooves, a ratchet mechanism, or other suitable control devices. In this regard, when device 200 is used for vaginal examinations or colposcopy, device 200 may, in many respects, resemble a prior art speculum.

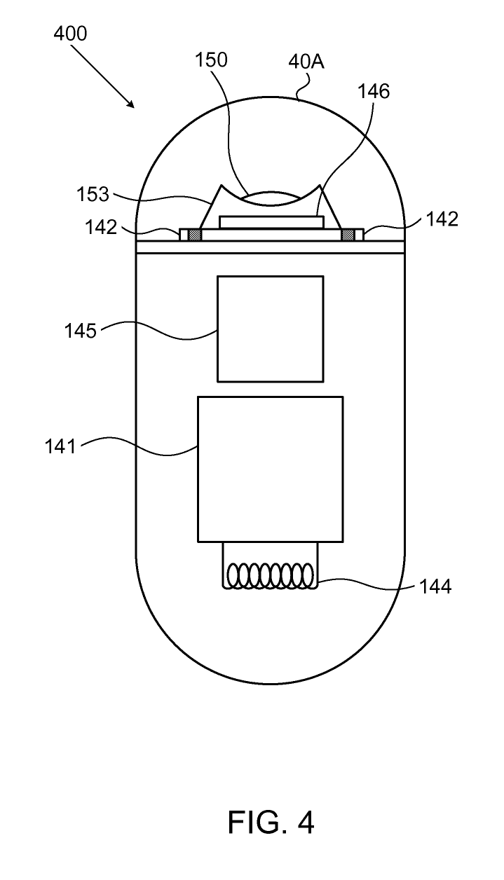

According to one embodiment, a sensing unit 210, one embodiment of which will be exemplified in FIG. 4, is attached to or included within one of arms 22a or 22b of device 200. Typically, as shown in FIG. 2, the sensing unit 210 is located at a distal end of the arm 22a or 22b so as to be in easy contact with or to have a closer view of the tissue being examined. As shown in FIG. 2, sensing unit 210 is located at a distal end of lower arm 22b so as to have a more stable viewpoint during a gynecological or colposcopy procedure, as lower arm 22b typically moves less during a gynecological or colposcopy procedure than does upper arm 22a, which is typically manipulated, i.e., by rotation or vertical movement, with respect to lower arm 22b during such procedures.

The sensing unit 210 may be positioned and/or shaped so as to enable painless and easy insertion into a patient's body orifice. For example, sensing unit 210 may include a curved or dome shaped leading end so as to push aside collapsed tissue and enable tissue to easily glide over the leading tip of the sensing unit 210.

Sensing unit 210 may include a sensing module (such as a pH sensor, temperature sensor, electrical impedance sensor, pressure sensor, image sensor and/or other suitable in-vivo sensors), an internal power source such as a battery, and a transmitter to transmit sensed signals. According to one embodiment, sensing unit 210 may be connected by a wire 27, typically running through handle 24 to external devices such as receiver 12 or monitor 18. Alternatively, there is no wire to external devices such as receiver 12 or monitor 18, and the transmitter transmits sensed signals wirelessly to external devices. In certain embodiments, sensing unit 210 does not comprise a transmitter, and a transmitter may be separate and distinct from sensing unit 210.

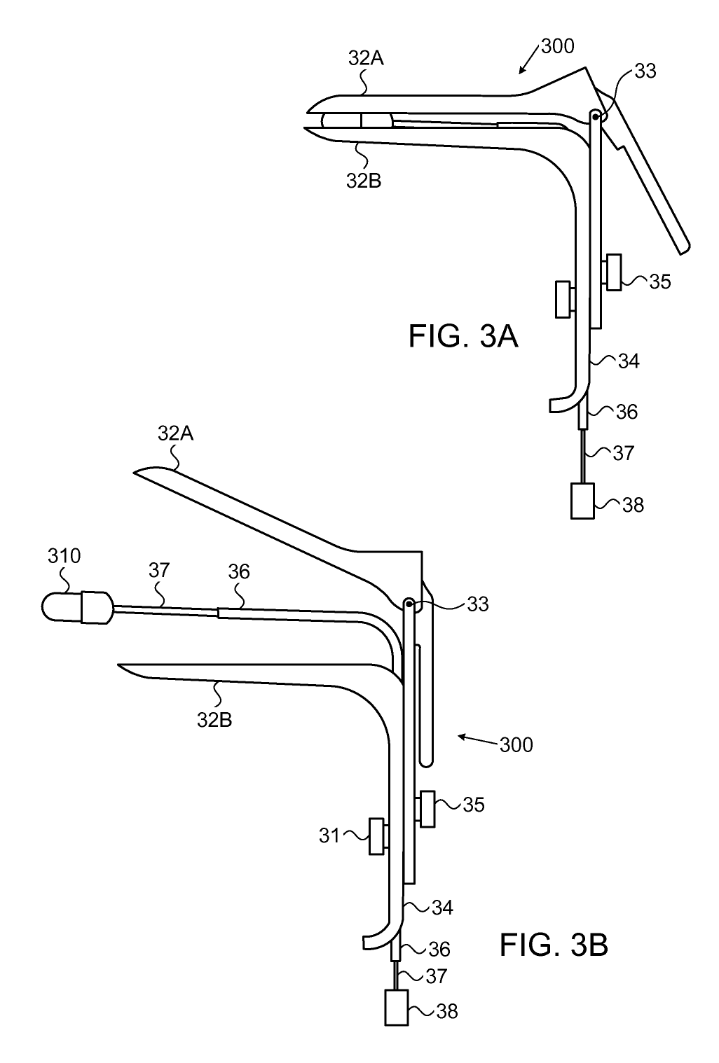

Reference is now made to FIGS. 3A-B, which depict devices operative according to embodiments of the present invention. In FIG. 3A, a device 300, which may be a speculum (such as that described in FIG. 2) but which may be another suitably-shaped insertion device, is in its closed position, wherein arms 32a and 32b are parallel to each other and the tips of arms 32a and 32b are in close proximity. According to one embodiment, a sensing unit 310 is concealed within device 300 while the device 300 is in its closed position. According to one embodiment, the device 300 is inserted into a body orifice (such as the vagina) in its closed position, is next opened inside the orifice (as shown in FIG. 3B), by raising upper arm 32a with respect to lower arm 32b and by increasing the angle between arms 32a and 32b, and is then secured in that adjusted position using mechanism 35.

Once device 300 is in such an open position, sensing unit 310 can then be moved upward to be spaced away from both speculum arms 32a and 32b and can be moved forward, i.e., outward of device 300, to protrude from the distal end of open device 300 into the body lumen being examined, using a mechanism, for example as depicted in FIG. 3B. Sensing unit 310 may be manipulated through use of a rigid but flexible holder rod 37 that grips sensing unit 310 at the distal end of rod 37. Rod 37 retains sensing unit 310 at its distal end by any known means, such as mechanical means, for example friction, snap fit or vacuum, magnetic means, electrical means, etc. Rod 37 extends backwards from its distal position between speculum arms 32a and 32b, through handle 34, to a proximal position at the base of handle 34, where it can be manipulated by a user. Typically, as shown in FIGS. 3A and 3B, rod 37 extends generally vertically through handle 34 at its proximal end, and rod 37 extends generally horizontally between speculum arms 32a and 32b at its distal end. Rod 37 may preferably be composed of a stiff material that flexes but retains its stiffness once it returns to its straight form.

In one embodiment, a guide tube 36, having an annular cavity formed therethrough, extends from below handle 34, through handle 34 and into the gap formed between arms 32a and 32b. Handle 34 has an internal vertical passageway that is sized such that guide tube 36 is easily longitudinally movable within and relative to handle 34. As shown in FIGS. 3A and 3B, guide tube 36 extends generally vertically through handle 34 at its proximal end, and guide tube 36 extends generally horizontally between speculum arms 32a and 32b at its distal end. Rod 37 extends completely through guide tube 36, and the annular cavity of guide tube 36 is sized with respect to rod 37 such that rod 37 is easily longitudinally movable within and relative to guide tube 36. Guide tube 36 is typically movable within device 300 by vertical movement with respect to lower arm 32b and handle 34, e.g., from a lowered position as shown in FIG. 3A to a raised position as shown in FIG. 3B.

In operation, in order to change the position of sensing unit 310, a user may grasp extender handle 38 at the proximal end of rod 37 and thereby push sensing unit 310 forward. Rod 37 is pushed forward through guide tube 36 into the lumen or is pulled back through guide tube 36 into device 300, pushing or pulling sensing unit 310 along with it to accomplish forward or backward movement of sensing unit 310. To move sensing unit upward with respect to lower arm 32b, guide tube 36 may be moved upward with respect to handle 34 such that the distal portion thereof, which is oriented horizontally, is spaced vertically from lower arm 32b. Handle 34 may include one or more mechanisms, such as mechanism 31, which may be a button, knob, bolt, groove, ratchet mechanism or other suitable control device, for securing guide tube 36 and rod 37 in their respective adjusted positions.

According to one embodiment, device 300 may include a mechanism within arm 32a which is in communication (for example, mechanical or electrical communication) with hinge 33 such that, upon increasing of the angle between arms 32a and 32b, the mechanism slides sensing unit 310 out of the arm 32a or at least within arm 32a but towards the distal end thereof. According to another embodiment, a mechanism for sliding sensing unit 310 towards the distal end of arm 32a may be controlled by a user, for example through controls on handle 34.

Upon opening of the arms of device 300 or upon increasing of the angle between arms 32a and 32b of device 300, rod 36 and attached sensing unit 310 may be pushed forward out of device 300 into the body orifice and manipulated within the body orifice for better viewing thereof. For example, rod 37 may be turned about its axis to enable sensing unit 310 to have different viewing angles. According to some embodiments, rod 36 may be twisted to bring sensing unit 310 to typically inaccessible areas within the orifice.

Rod 37 may be made of a suitable material such as plastic or a rigid yet flexible material to allow rigidity and flexibility as required. Rod 37 also may include wires to electrically connect unit 310 with external devices. Alternatively, there may not be any wire within rod 37 to provide a physical connection to external devices such as receiver 12 or monitor 18, and instead the transmitter transmits sensed signals wirelessly to external devices. The sensing unit 310 may be retrieved from the body orifice together with or separately from the device 300.

According to some embodiments, sensing unit 310 may be used for performing activities such as taking physical biopsies, spraying dye on tissues, ligating or cauterizing lesions, excising polyps and other in vivo activities, apart from or in addition to the sensing functions described above, for example, as described in U.S. Pat. No. 6,936,003 (Iddan) and U.S. Patent Application Publication No. 2008/0199065 (Swain).

According to one embodiment, the sensing unit 310 may be connected to a mechanism which is separate from the device 300. For example, sensing unit 310 may be attached to element 36. According to one embodiment, element 36 is a rod or hollow tube that is rigid enough to be pushed through, for example, handle 34 or through a body orifice. Rod 36 may also need to be flexible enough to be able to follow contours of the handle or body orifice. In one embodiment, rod 37 and guide tube 36 may be separate from handle 34 and inserted therein.

According to certain embodiments of the invention, sensing unit 310 and/or device 300 are single-use articles, whose sterility is ensured for in every procedure, making the procedure safer for the patient. In such embodiments, it is preferable that single use sensing unit 310 and/or device 300 be composed of or made from materials that are not expensive, such that disposal of such articles are a single use will not impose a cost burden upon the user.

Reference is now made to FIG. 4 which depicts an imaging unit according to an embodiment of the present invention. According to one embodiment, the sensing unit described above is an imaging unit 400.

In contrast to other medical instruments, such as a colposcope, that perform their examinations or inspections from outside the patient's body, the medical device as described herein is intended to allow the physician to perform an examination or inspection from inside the patient's body. Accordingly, in one embodiment, unit 400 has a rounded or dome shaped leading end or tip 40a. A dome shaped tip may enable easy separation of and passage of unit 400 through collapsed lumens, such as the vaginal cavity, GI tract and other bladders and tubes within the body. Additionally, a dome shaped leading end may serve to space the examined tissue from an optical system and/or illumination source so as to enable good lighting and viewing conditions for the examined tissue. According to other embodiments, unit 400 may assume another shape, for example, a spherical or rod shape, so as to be accommodated within the insertion device through which it is being introduced into the body lumen, as well as to accommodate for the anatomy of the lumen it is viewing.

According to one embodiment, unit 400 includes an imager 146, one or more illumination sources 142, a power source 145, and a transmitter 141. In some embodiments, device 400 may be implemented using a system similar to that described in U.S. Pat. No. 7,009,634 to Iddan et al., which is incorporated herein by reference.

In some embodiments, unit 400 may include one or more illumination sources 142, for example one or more Light Emitting Diodes (LEDs), “white LEDs”, or other suitable light sources. Illumination sources 142 may, for example, illuminate a body lumen or cavity being imaged and/or sensed. Illumination sources 142 may be white light, i.e., full wavelength spectrum, or may be other than white light. For example, illumination sources 142 may illuminate in a specific wavelength or range of wavelengths, or may have one or more color filters to filter out all but the desired color or colors of light.

For example, illumination sources 142 may illuminate in only blue light or suitable filters may allow only blue light to pass through, in order to be able to notice certain characteristics of the tissue that may be less noticeable when illuminated in other specific wavelengths or groups of wavelengths, including white light. Other illumination sources 142 which may illuminate in specific wavelengths other than blue may be used, e.g. Infrared (IR), near infrared (NIR), etc. Also, an optical biopsy may be performed by illuminating in more than one selected wavelength (along with the imager comprising different filters), and this may assist in determining whether the tissue imaged is cancerous or benign. This may be done by comparing, e.g., via spectroscopic analysis, the colors of the image of the tissue to the wavelengths illuminating the tissue.

An optical system 150, including, for example, one or more optical elements, such as one or more lenses or composite lens assemblies, one or more suitable optical filters, or any other suitable optical elements, may be included in unit 400, typically positioned on an optical tower 153, and may aid in focusing reflected light onto imager 146, focusing illuminated light, and/or performing other light processing operations.

In some embodiments, illumination source(s) 142 may illuminate continuously, or substantially continuously, for example, not necessarily upon-demand, or not necessarily upon a triggering event. In some embodiments, for example, illumination source(s) 142 may illuminate a pre-defined number of times per second (e.g., two or four times), substantially continuously, e.g., through out a gynecological examination procedure; or in a periodic manner, for example when unit 400 is used for monitoring an in-vivo site, an intermittent manner, or an otherwise non-continuous manner.

In some embodiments, unit 400 may include an in-vivo imager 146, for example, a video camera, which may capture and transmit images of a body lumen, for example, a uterus, the GI tract, a urinary duct or bladder, etc. Other lumens and/or body cavities may be imaged and/or sensed by unit 400. In some embodiments, imager 146 may include, for example, a Charge Coupled Device (CCD) camera or imager, a Complementary Metal Oxide Semiconductor (CMOS) camera or imager. In some embodiments, imager 146 may record images in a high definition resolution format, such as 1280×720 or 1920×1080 frame size, and may utilize a high definition compression format, such as HDV (“High Definition Video”) and AVCHD (“Advanced Video Codec High Definition”), both of which are well known in the field.

In some embodiments, imager 146 may acquire in-vivo images continuously, substantially continuously, or in a non-discrete manner, for example, not necessarily upon-demand, or not necessarily upon a triggering event.

In some embodiments, unit 400 may communicate with an external receiving and display system to provide display of data, control, or other functions. Transmitter 141 may operate using radio waves or other known methods of wireless transmission, but in some embodiments, transmitter 141 may transmit/receive data via, for example, wire, optical fiber and/or other suitable methods. Transmitter 141 may include, for example, a transmitter module or sub-unit and a receiver module or sub-unit, or an integrated transceiver or transmitter-receiver.

Transmitter 141 may transmit images to, for example, external transceiver or receiver/recorder (e.g., through one or more antennas, either individually or in an array), which may send the data to a processor and/or to storage unit. Transmitter 141 may include any suitable transmitter able to transmit image data, including high definition format data, other sensed data, and/or other data (e.g., control data) to a receiving device. For example, in some embodiments, transmitter 141 may include an ultra low power Radio Frequency (RF) high bandwidth transmitter, possibly provided in Chip Scale Package (CSP).

Transmitter 141 may also be capable of receiving signals/commands, for example from an external transceiver. According to some embodiments, transmitter 141 may include a receiver which may receive signals (e.g., from outside the patient's body), for example, through the antenna 144 or antenna array or through a different receiving element. According to some embodiments, signals or data may be received by a separate receiving device in unit 400.

In some embodiments, transmitter 141 may transmit image data continuously, or substantially continuously, for example, not necessarily upon-demand, or not necessarily upon a triggering event. In some embodiments, imager 146 may be operationally connected to transmitter 141.

Transmitter 141 may also include control capability, although control capability may be included in a separate component, e.g., a dedicated processor. In some embodiments, transmitter 141 may include a processing unit or processor or controller, for example, to process signals and/or data generated by imager 146, including those of high definition format. In another embodiment, the processing unit may be implemented using a separate component within unit 400, e.g., a controller or processor (not shown), or may be implemented as an integral part of imager 146, transmitter 141, or another component, or may not be needed. The processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In some embodiments, for example, the processing unit or controller may be embedded in or integrated with transmitter 141, and may be implemented, for example, using an ASIC.

Power may be provided to unit 400 using an internal battery, an internal power source, or a wireless system able to receive power. Other embodiments may have other configurations and capabilities. For example, components may be distributed over multiple sites or units, and control information or other information may be received from an external source.

Power source 145 may include one or more batteries or power cells. For example, power source 145 may include silver oxide batteries, lithium batteries, other suitable electrochemical cells having a high energy density, or the like. Other suitable power sources may be used. For example, power source 145 may receive power or energy from an external power source (e.g., an electromagnetic field generator), which may be used to transmit power or energy to in-vivo unit 400. In some embodiments, power source 145 may provide power to one or more components of unit 400, for example, upon-demand, or upon a triggering event or an external activation.

Embodiments of unit 400 are typically self-contained. For example, unit 400 may be a capsule or other unit where all the components are substantially contained within a housing or shell, and where unit 400 does not require any external wires or cables to, for example, receive power or transmit information.

In some embodiments, the components of unit 400 may be enclosed within a housing or shell, which may be, e.g., capsule-shaped, oval, or having other suitable shapes. The housing or shell may be substantially transparent or semi-transparent, and/or may include one or more portions, windows or domes which may be substantially transparent or semi-transparent. For example, one or more illumination source(s) 142 within unit may illuminate a body lumen through a transparent or semi-transparent portion, window or dome 152; and light reflected from the body lumen may enter the unit 400, for example, through the same transparent or semi-transparent portion, window or dome, or, optionally, through another transparent or semi-transparent portion, window or dome, and may be received by optical system 150 and/or imager 146. In some embodiments, for example, optical system 150 and/or imager 146 may receive light, reflected from a body lumen, through the same window or dome through which illumination source(s) 142 illuminate the body lumen.

According to some embodiments, the optical system may include a micro-motor (such as motors manufactured by Nanomotion Inc.) for enabling the optical system different magnifications.

Embodiments of the invention include a method for examining the cervix. According to one embodiment, the method includes inserting into the body orifice an imaging unit which captures images of the cervix tissue and transmits the images to an external unit to be viewed by the physician on a monitor screen. According to some embodiments of the invention, the step of imaging may be assisted by appropriate software to automatically identify pathologies or anatomical structures. According to other embodiments, the imaging may be aided by additional sensing modules, such as modules mentioned above and other modules, for example, optical biopsy techniques (for example as described in U.S. Patent Application Publication No. 2005/0004474 Iddan).

According to one embodiment, a sensing unit may be introduced into a body lumen (such as a uterus or GI tract), for example, by an introducing device as described above. The sensing unit may be immobilized at an in vivo location for monitoring the in vivo site (for example, to monitor vaginal dilution during pregnancies, to monitor a site in vivo post surgery and other).

It will be appreciated by persons skilled in the art that the present invention is not limited by what has been particularly shown and described hereinabove. Rather the scope of the invention is defined by the claims that follow:

1. A device for gynecological examination, comprising:
a vaginal speculum comprising:
an upper blade having a distal end and a proximal end,
a lower blade with a distal end and a proximal end,
said blades having means on their respective proximal ends for moveably interconnecting said proximal ends for relative movement between said first blade and said second blade, and
a handle attached to said lower blade; and
a sensing unit movably positioned between said upper and lower blades, said sensing unit comprising a sensing means for sensing data of a body lumen; and
a sensing unit movement mechanism passing through said handle and having a distal end attached to said sensing unit, said sensing unit movement mechanism enabling movement of said sensing unit relative to said upper and lower blades via remote actuation of a proximal end of said sensing unit movement mechanism.

2. The device of claim 1 wherein said sensing means comprises means for sensing pH, temperature, electrical impedance, pressure or image data.

3. The device of claim 2 wherein said sensing means comprises an image sensor having an illumination source, an optical apparatus and an imager.

4. The device of claim 1 wherein said sensing means comprises a transmitter for transmitting sensed data to a remote receiver.

5. The device of claim 1 wherein said sensing unit movement mechanism comprises an elongated connection element having proximal and distal ends, said distal end configured for attachment to said sensing unit, and said proximal end configured for manipulation by a user.

6. The device of claim 5 wherein said elongated connection element extends through said handle.

7. The device of claim 5,
wherein application of a force by a user on the proximal end of the elongated connection element in the proximal direction creates outward motion of said sensing unit relative to said upper and lower blades, and
wherein application of a force by a user on the proximal end of the elongated connection element in the distal direction creates inward motion of said sensing unit relative to said upper and lower blades.

8. The device of claim 5 further comprising an elongated guide tube having proximal and distal ends, said distal end situated between said upper and lower blades, and said proximal end configured for manipulation by a user.

9. The device of claim 8 wherein said guide tube is movable within said handle in the direction of said handle.

10. The device of claim 8 wherein connection element extends through and is movable within said guide tube.

11. The device of claim 1 further comprising means for attaching said sensing unit to said distal end of said sensing unit movement mechanism.

12. A system for gynecological examination, comprising:
the device of claim 1;
a receiver for receiving signals representing sensed data transmitted from said device; and
a means for presenting said sensed data of a body lumen to a user.

13. The system of claim 12 wherein said receiver is remote and not physically connected to said device.

14. The system of claim 12 wherein said sensed data is image data and said means for presenting comprises a display for displaying to a user said sensed data.

15. The system of claim 12 wherein said means for presenting further comprises a processor for processing sensed data.

16. The system of claim 12 wherein said device further comprises a transmitter for transmitting from said device signals representing sensed data.

17. A method of gynecological examination, comprising:
utilizing the device of claim 1 to sense data of a patient's vaginal region,
transmitting the sensed data to a receiver,
processing the received data, and
presenting the processed data to a user.
